(19) United States
(12) Patent Application Publication
(10) Pub. No.: US 2003/0204172 A1
(43) Pub. Date: Oct. 30, 2003

(54) Aspiration system

(76) Inventor: Dennis L. Steppe, Corona, CA (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/132,799

(22) Filed: Apr. 25, 2002

(51) Int. Cl. A61M 1/00

(52) U.S. Cl. 604/319

(57) ABSTRACT

A dual pump aspiration system having both a vacuum level control loop and a flow rate control loop. The system can be operated either as a vacuum priority system or a flow rate priority system and uses a vacuum chamber of variable volume within the vacuum pathways.

### BACKGROUND OF THE INVENTION

During small incision surgery, and particularly during ophthalmic surgery, small probes are inserted into the operative site to cut, remove or otherwise manipulate tissue. During these surgical procedures, the surgical site typically is flushed with an irrigating solution and the irrigating solution and tissue is aspirated from the surgical site. The types of aspiration system used, prior to the present invention, where generally characterized as either flow controlled or vacuum controlled, depending upon the type of pump used in the system, and each type of system has certain advantages.

Vacuum controlled aspiration systems are operated by setting a desired vacuum level, which the system seeks to maintain. Flow rate information is unavailable. Vacuum controlled aspiration systems typically use a venturi or diaphragm pump. Vacuum controlled aspiration systems offer the advantages of quick response times, control of decreasing vacuum levels and good fluidic performance while aspirating air, such as during an air/fluid exchange procedure. Disadvantages of such systems are the lack of flow information resulting in high flows during phacoemulsification/fragmetation coupled with a lack of occlusion detection. Vacuum controlled systems are difficult to operate in a flow controlled mode because of the problem of non-invasively measuring flow in real time.

Flow controlled aspiration systems are operated by setting a desired aspiration flow rate for the system to maintain. Flow controlled aspiration systems typically use a peristaltic, orbital or vane pump. Flow controlled aspiration systems offer the advantages of stable flow rates and automatically increasing vacuum levels under occlusion. Disadvantages of such systems are relatively slow response times, undesired occlusion break responses when large compliance components are used and vacuum can not be linearly decreased during tip occlusion. Flow controlled systems are difficult to operate in a vacuum controlled mode because time delays in measuring vacuum can cause instability in the control loop, reducing dynamic performance.

One surgical system currently commercially available, the Millennium from Storz Instrument Company, contains both a vacuum controlled aspiration system (using a venturi pump) and a flow controlled aspiration system (using an orbital pump). The two pumps can not be used simultaneously, and each pump requires separate aspiration tubing and cassette.

Another currently available system, the ACCURUS® system from Alcon Laboratories, Inc., contains both a venturi pump and a peristaltic pump that operate in series. The venturi pump aspirates material from the surgical site to a small collection chamber. The peristaltic pump pumps the aspirate from the small collection chamber to a larger collection bag. The peristaltic pump does not provide aspiration vacuum to the surgical site. Thus, the system operates as a vacuum controlled system.

Accordingly, a need continues to exist for a surgical system that operates in both vacuum controlled and flow controlled modes.

### BRIEF SUMMARY OF THE INVENTION

The present invention improves upon prior art by providing a dual pump aspiration system having both a vacuum level control loop and a flow rate control loop. The system can be operated either as a vacuum priority system or a flow rate priority system and uses a vacuum chamber of variable volume within the vacuum pathways. The vacuum chamber has an inner housing and an outer housing that are movable relative to each other.

Accordingly, an objective of the present invention to provide a dual pump aspiration system.

Another objective of the present invention to provide an aspiration system having both a vacuum level control loop and a flow rate control loop.

A further objective of the present invention to provide an aspiration control system and method that can be operated either as a vacuum priority system or a flow rate priority system.

A further objective of the present invention to provide an aspiration control system having a vacuum chamber of variable volume.

Other objectives, features and advantages of the present invention will become apparent with reference to the drawings, and the following description of the drawings and claims.

### BRIEF DESCRIPTION OF THE DRAWINGS

FIG. 1 is a schematic diagram of a first embodiment of the dual mode system of the present invention.

FIG. 2 is a schematic diagram of a second embodiment of the dual mode system of the present invention.

### DETAILED DESCRIPTION OF THE INVENTION

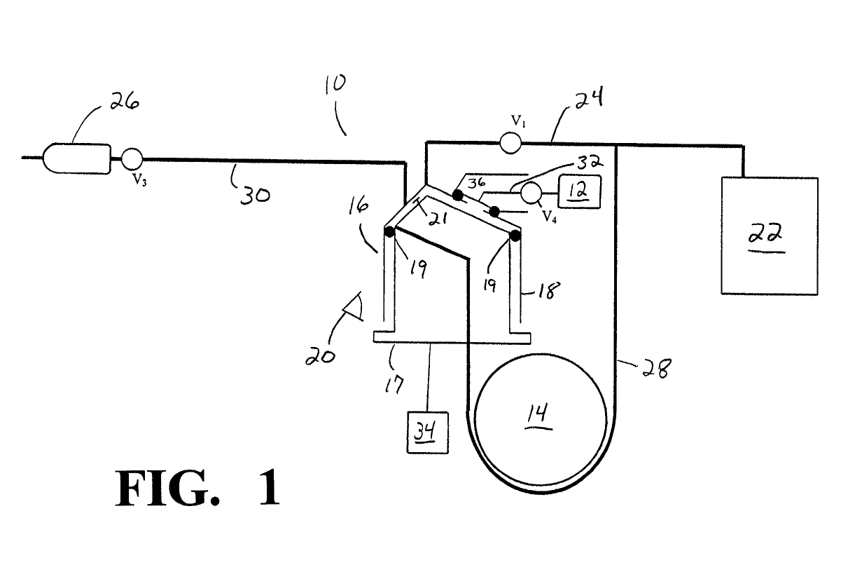

As best seen in FIG. 1, system 10 of a first embodiment of the present invention generally contains vacuum pump 12, flow pump 14, variable volume chamber (VVC) 16, optical sensor 20, collection chamber 22 and handpiece 26. Vacuum pump 12 may be any 67 suitable pump, such as a diaphragm pump, a vane pump, an orbital pump or a peristaltic pump, but a venturi pump is preferred. Flow pump 14 may be any suitable pump, such as a venturi pump, a diaphragm pump, a vane pump or an orbital pump, but a peristaltic pump is preferred. VVC 16 contains inner housing 17 and outer housing 18 separated by seals 19. Inner housing 17 is sized to reciprocate within outer housing 18 by, for example, stepper motor or other driver 34, so as to define variable interior volume 21. VVC 16 may be of any suitable shape in cross-section, but elliptical is preferred. VVC 16 preferably is made from optically clear, medical grade thermoplastic. Optical sensor 20 may be any suitable device for measuring the relative position of inner housing 17 and outer housing 18. Interior volume 21 is fluidly connected to collection chamber 22 through outer housing 18, line 24 and valve V₁. Flow pump 14 is fluidly connected to interior volume 21 and collection chamber 22 through inner housing 17, line 28 and line 24. Handpiece 26 is fluidly connected to interior volume 21 through outer housing 18, line 30 and valve V₃. Vacuum pump 12 is fluidly connected to interior volume 21 through outer housing 18, line 32 and valve V₄.

The connections to vacuum pump 12 and flow pump 14, as well as VVC 16, collection chamber 22, fluid lines to VVC 16 and valves V₁ and V₃ preferably are all contained within or withon a common housing or cassette for attachment to a control console (not shown) using latch mechanism 36. Vacuum pump 12, flow pump 14 and valve V₄ are preferably contained in the control console.

In use, system 10 of the present invention is first primed by opening valve V₃ and motor 34 drawing out inner housing 17 from within outer housing 18, thereby increasing the size of interior volume 21 and drawing fluid into interior volume 21 through handpiece 26, line 30 and outer housing 18. When inner housing 17 reaches a predetermined location, as sensed by optical sensor 20, movement of inner housing 17 stops, valve V₃ is closed, valve V₄ is opened and flow pump 14 is started, thereby draining fluid from interior volume 21 and into collection chamber 22 through line 28 and drawing air into interior volume 21 through valve V₄. Operation of flow pump 14 is stopped prior to draining the contents of interior volume 21, valve V₄ is closed and valve V₁ is opened. Motor 34 then moves inner housing 17 into outer housing 18, forcing the remaining air and fluid within interior volume 21 into collection chamber 22 through line 24.

To operate system 10 in a vacuum control mode, valves V₃ and V₄ are closed and 67 valve V₁ is opened. Inner housing 17 is driven by motor 34 into outer housing 18 to a “home” or fully closed position. Valve V₁ is closed and valve V₄ is opened. Inner housing 17 is drawn out of outer housing 18 to a predetermined position so as to provide the optimum volume for interior volume 21. Vacuum pump 12 is then set to the desired aspiration vacuum level and valve V₃ is opened, allowing fluid to flow through handpiece 26 and line 30 into interior volume 21. When the fluid level in interior volume 21 reaches its maximum, flow pump 14 activates to drain fluid out of interior volume 21 and into collection chamber 22.

To operate system 10 in a flow control mode, valves V₃ and V₄ are closed and valve V₁ is opened. Inner housing 17 is driven by motor 34 into outer housing 18 to a “home” or fully closed position. Valve V₁ is closed. Fluid flow rate may be maintained in two alternative methods. In the first method, valve V₃ is opened and inner housing 17 is drawn from outer housing 18 by motor 34 at a predetermined speed so as to cause a vacuum to be created in interior volume 21 and thereby provide the desired flow rate through handpiece 26 and line 30. When inner housing 17 reaches the full extent of its travel (as sensed by sensor 20), valve V₃ is closed and valve V₁ is opened. Inner housing 17 is then forced back within outer housing 18 by motor 34, thereby pressurizing interior volume 21 and forcing fluid out of interior volume 21 and into collection chamber 22 through line 24 until inner housing 17 reaches the “home” position. The cycle discussed above is then repeated until the surgical procedure is completed.

In a second flow control method, inner housing 17 is drawn out of outer housing 18 by motor 34 to a predetermined location so as to provide an optimum volume for interior volume 21. Flow pump 14 is activated and run at a speed sufficient to provide the required fluid flow rate into from interior volume 21 to collection chamber 22 through line 28.

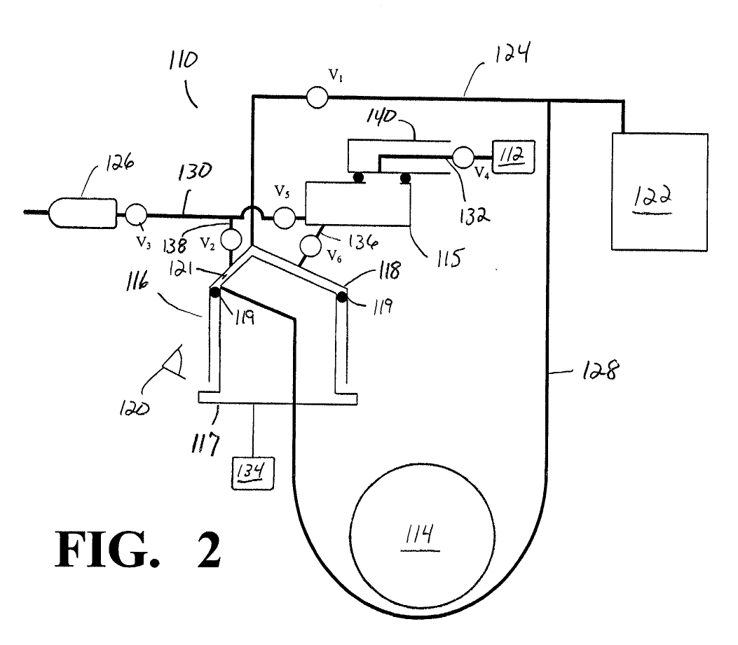

As best seen in FIG. 2, system 110 of a second embodiment of the present invention generally contains vacuum pump 112, flow pump 114, variable volume chamber (VVC) 116, fixed volume chamber (FVC) 115, optical sensor 120, collection chamber 122 and handpiece 126. Vacuum pump 112 may be any suitable pump, such as a diaphragm pump, a vane pump, an orbital pump or a peristaltic pump, but a venturi pump is preferred. Flow pump 114 may be any suitable pump, such as a venturi pump, a diaphragm pump, a vane pump or an orbital pump, but a peristaltic pump is preferred. VVC 116 contains inner housing 117 and outer housing 118 separated by seals 119. Inner housing 117 is sized to reciprocate within outer housing 118 by, for example, stepper motor or other driver 134, so as to define variable interior volume 121. VVC 116 may be of any suitable shape in cross-section, but elliptical is preferred. VVC 116 and FVC 115 preferably are made from optically clear, medical grade thermoplastic. Optical sensor 120 may be any suitable device for measuring the relative position of inner housing 117 and outer housing 118. Interior volume 121 is fluidly connected to collection chamber 122 through outer housing 118, line 124 and valve V₁. Flow pump 114 is fluidly connected to interior volume 121 and collection chamber 122 through inner housing 117, line 128 and line 124. Handpiece 126 is fluidly connected to interior volume 121 through outer housing 118, line 138, valve V₂, line 130 and valve V₃. Vacuum pump 112 is fluidly connected to interior volume 121 through outer housing 118, line 136, valve V₆, FVC 115, line 132 and valve V₄. FVC 115 is fluidly connected to handpiece 126 through valve V₅ and line 130.

The connections to vacuum pump 112 and flow pump 114, as well as VVC 116, FVC 115, collection chamber 122, fluid lines to VVC 116 and FVC 115 and valves V₁ V₂, V₃ V₅ and V₆ preferably are all contained within or withon a common housing or cassette for attachment to a control console (not shown) using latch mechanism 140. Vacuum pump 112, flow pump 114 and valve V₄ are preferably contained in the control console.

In use, system 110 of the present invention is first primed by opening valve valves V₃, V₅ and V₆ and motor 134 drawing out inner housing 117 from within outer housing 118, thereby increasing the size of interior volume 121 and drawing fluid into interior volume 121 through handpiece 126, lines 130 and 136 and outer housing 118. When inner housing 117 reaches a predetermined location, as sensed by optical sensor 120, or after a predetermined time, valves V₅ and V₆ are closed and valve V₂ is opened. Outward movement of inner housing 117 continues until an appropriate fluid level in interior volume 121 is reached or the appropriate amount of time has pasted at which time valves V₃ and V₂ are closed, valve V₄ is opened and flow pump 114 is started, thereby draining fluid from interior volume 121 and into collection chamber 122 through line 128 and drawing air into interior volume 121 through valve V₄. Operation of flow pump 114 is stopped prior to draining the contents of interior volume 121, valve V₄ is closed and valve V₁ is opened. Motor 134 then moves inner housing 117 into outer housing 118, forcing the remaining fluid within interior volume 121 into collection chamber 122 through valve V₁ and line 124.

To operate system 110 in a vacuum control mode, valves V₃, V₅ and V₆ are closed. and valve V₁ is opened. Inner housing 117 is driven by motor 134 into outer housing 118 to a “home” or fully closed position. Valve V₁ is closed and valve V₄ is opened. Inner housing 117 is drawn out of outer housing 118 to a predetermined position so as to provide the optimum volume for interior volume 121. Vacuum pump 112 is then set to the desired aspiration vacuum level and valves V₂, V₃ and V₆ are opened, allowing fluid to flow through handpiece 126, lines 130 and 138 and valve V₂ into interior volume 121 and through line 136 into FVC 115. In this manner, FVC 115 acts as a fluid capacitor, providing a fluidic buffer between vacuum pump 112 and VVC 116. When the fluid level in interior volume 121 reaches its maximum, flow pump 114 activates to drain fluid out of interior volume 121 and into collection chamber 122.

To operate system 110 in a flow control mode, valves V₅ and V₆ are closed and valves V₂ and V₃ are opened and inner housing 117 is driven by motor 134 into outer housing 118 to a “home” or fully closed position. Valve V₁ is closed. This flow rate may be maintained in two alternative methods. In the first method, inner housing 117 is drawn from outer housing 118 by motor 134 at a predetermined speed so as to provide the desired flow rate through handpiece 126 and lines 130 and 138. When inner housing 117 reaches the full extent of its travel (as sensed by sensor 120), valve V₂ is closed and valve V₁ is opened. Inner housing 117 is then forced back within outer housing 118 by motor 134, thereby forcing fluid out of interior volume 121 and into collection chamber 122 through line 124 until inner housing 117 reaches the “home” position. The cycle discussed above is then repeated until the surgical procedure is completed.

In a second flow control method, inner housing 117 is drawn out of outer housing 118 by motor 134 to a predetermined location so as to provide an optimum volume for interior volume 121. Flow pump 114 is activated and run at a speed sufficient to provide the required fluid flow rate into from interior volume 121 to collection chamber 122 through line 128.

One skilled in the art will recognize that other methods of operating systems 10 and 110 are possible.

While certain embodiments of the present invention have been described above, these descriptions are given for purposes of illustration and explanation. Variations, changes, modifications and departures from the systems and methods disclosed above may be adopted without departure from the scope or spirit of the present invention.

I claim:

1. An aspiration system, comprising:
a) a chamber having a variable volume;
b) a vacuum pump fluidly connected to the chamber; and
c) a flow pump fluidly connected to the chamber.

2. The aspiration system of claim 1 further comprising a means for varying the volume of the chamber.

3. The aspiration system of claim 1 further comprising a chamber having a fixed volume fluidly connected between the variable volume chamber and the vacuum pump.

4. The aspiration system of claim 1 wherein the variable volume chamber is elliptical in cross-section.

5. The aspiration system of claim 1 wherein the variable volume chamber has a inner housing and an outer housing and the inner housing reciprocates within the outer housing.

6. The aspiration system of claim 5 wherein the flow pump is fluidly connected to the inner housing.

7. The aspiration system of claim 5 wherein the inner housing is reciprocated within the outer housing so as to create a vacuum within the variable volume chamber.

8. The aspiration system of claim 5 wherein the inner housing is reciprocated within the outer housing so as to pressurize the variable volume chamber.

9. An aspiration system, comprising:
a) a chamber having a variable volume located between an inner housing and an outer housing;
b) a means for reciprocating the inner housing within the outer housing;
c) a vacuum pump fluidly connected to the chamber; and
d) a flow pump fluidly connected to the chamber.

10. The aspiration system of claim 9 further comprising a chamber having a fixed volume fluidly connected between the variable volume chamber and the vacuum pump.

11. The aspiration system of claim 9 wherein the variable volume chamber is elliptical in cross-section.

12. The aspiration system of claim 9 wherein reciprocation of the inner housing within the outer housing creates a vacuum within the variable volume chamber.

13. The aspiration system of claim 9 wherein the flow pump is fluidly connected to the inner housing.

14. The aspiration system of claim 9 wherein reciprocation of the inner housing within the outer housing pressurizes the variable volume chamber.
